(19) United States
(12) Plant Patent Application Publication
(10) Pub. No.: US 2008/0282435 P1
(43) Pub. Date: Nov. 13, 2008

(54) Saxifraga plant named 'Rockred'

(75) Inventor: Martien Everett Gutter, Enkhuizen (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/800,857

(22) Filed: May 8, 2007

(51) Int. Cl. A01H 5/00

(52) U.S. Cl. PLT/263.1

(57) ABSTRACT

A new Saxifraga plant, particularly distinguished by its red flower color, early flowering and compact plant habit.

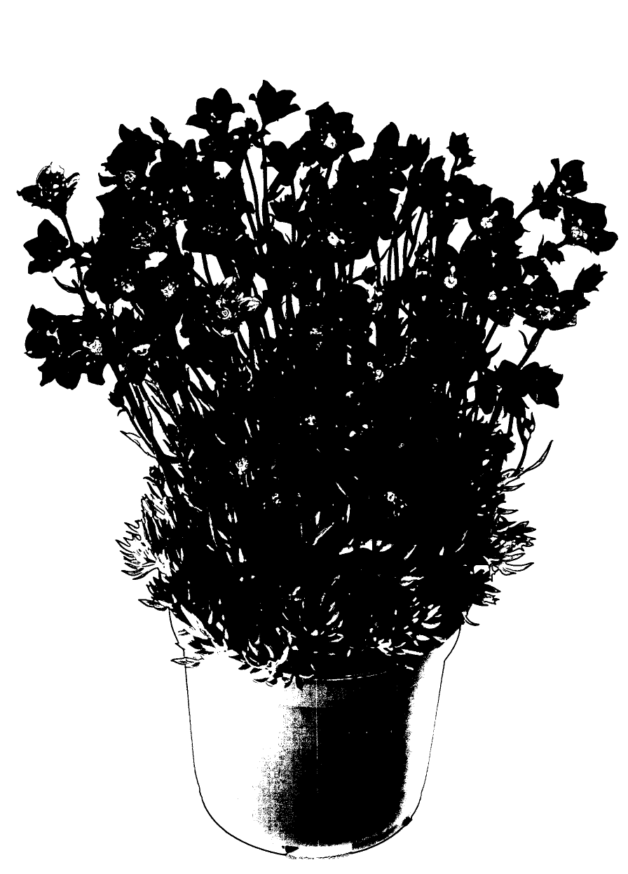

### LATIN NAME OF THE GENUS AND SPECIES OF THE PLANT CLAIMED

Saxifraga×arendsii

### VARIETAL DENOMINATION

‘Rockred’

### BACKGROUND OF THE NEW PLANT

The present invention comprises a new distinct cultivar of Saxifraga, botanically known as Saxifraga×arendsii.

The new Saxifraga is a product of a planned breeding program conducted in Enkhuizen, Netherlands. The new Saxifraga cultivar has a compact plant habit, a red flower color and an early flowering.

The new Saxifraga is propagated by cuttings resulting from a single seedling. This seedling originates from a red flowering selection in Saxifraga×arendsii ‘Purperteppich’ (not patented), identified as number ‘D702.’ This selection was made through three cycles of half sib selection. The selection was done in the period April 2001 until March 2003 in Enkhuizen, The Netherlands. The seedling was selected in March 2003 in Enkhuizen.

The present cultivar has been repeatedly asexually reproduced by cuttings in Enkhuizen, Netherlands, since 2003. The distinctive characteristics of this new Saxifraga are stable and reproduced true to type in successive generations of asexual reproduction. It takes 23 to 29 weeks to produce a finished plant, depending on the temperature.

The new Saxifraga plant is a perennial in all climatic zones in the US.

### DESCRIPTION OF THE DRAWING

The new Saxifraga plant is illustrated by the accompanying photographic drawing which shows blooms, buds and foliage of the plant in full color, the color showing being as true as can be reasonably obtained by conventional photographic procedures.

### DESCRIPTION OF THE NEW CULTIVAR

The following detailed descriptions set forth the distinctive characteristics of this new Saxifraga. The data which defines these characteristics were collected from asexual reproductions carried out in Enkhuizen, Netherlands. The plant history was taken on plants of 30 weeks old. The instant plant was grown in an 11 cm container in a plastic tunnel. Color references are primarily to The R.H.S. Color Chart of The Royal Horticultural Society of London.

Plants of the new Saxifraga differ primarily from the plants of the originator ‘Purperteppich’ in the following characteristics:

Plants of the new variety have uniform red flowers and a compact growth habit, whereas plants of ‘Purperteppich’ have red, rose and white flowers, and a loose growth habit.

TABLE 1
DIFFERENCES BETWEEN THE NEW CULTIVAR ‘ROCKRED’
AND A SIMILAR CULTIVAR

| | ‘Rockred’ | ‘Peter Pan Red’ (Not patented) |
|---|---|---|
| Color of young flower | 60A | 63B |
| Time of flowering | 23-29 weeks | 26-32 weeks after cutting |
| | after cutting | |

The plant:
   Classification—botanical.—Saxifraga×arendsii.
Parentage:
   Originator selection.—‘Purperteppich’ (Not patented).
Propagation:
   Type of cutting.—Terminal cuttings.
   Time to initiate roots.—12-14 days at 18° C.
   Time to produce a rooted cutting.—5 weeks.
   Root description.—Fine, fibrous and white in color.
Plant description:
   General appearance.—Makes mats of rosettes. Each rosette develops several short branches, and one or more branched flower stems.
   Growth and branching habit.—Freely branching habit, freely flowering, vigorous.
   Plant height.—16 cm.
   Plant spread.—15 cm.
Lateral branches:
   Length.—3-6 cm.
   Diameter.—1.2 mm.
   Texture.—Smooth; pubescent.
   Internode length.—2-5 mm.
   Color.—145C.
The foliage:
   Arrangement.—Alternate.
   Shape of blade.—Three-lobed.
   Length, mature leaves.—0.8-1.3 cm.
   Width, mature leaves.—0.7-1.2 cm.
   Apex.—Acute.
   Base.—Acute.
   Margin.—Entire; the two outer lobes are sometimes incised.
   Texture.—Smooth, slightly pubescent.
   Venation pattern.—Palmate.
   Color of upper surface.—144A.
   Color of lower surface.—144B.
   Venation, upper surface.—145B.
   Venation, lower surface.—144A.
The petiole:
   Length.—0.8-1.2 cm.
   Width.—1.3 mm.
   Color upper surface.—144B.
   Color lower surface.—144C.
   Texture.—Smooth; slightly pubescent.
The flower:
   Inflorescence type.—Each rosette develops 1-3 branched flower stems. The flower stem is sometimes fasciated.
   Flower type.—Single, solitary; symmetry: radial symmetric.
   Number of branches per flower stem.—2-5.
   Number of flowers and flower buds per flower stem.—2-15.
   Length flower stem.—7-15 cm.
   Flower position.—At top of branch.
   Fragrance.—Absent.
   Flowering season.—Spring.
   Flower diameter.—1.2-2.2 cm.
   Lastingness of the bloom.—40-50 days.
   Lastingness of an individual flower.—3-6 days.
Flower buds:
   Bud length (at stage of showing color).—3 mm.
   Bud diameter.—3 mm.
   Bud shape.—Orbicular.
   Texture.—Smooth; pubescent, hairs have a purple tip (N79B).
   Color, just before opening.—146B, with 63A on apex.
Petals:
   Quantity.—5 per flower, imbricate. Some flowers develop no or very small petals.
   Shape.—Orbicular.
   Apex.—Rounded to acuminate.
   Base.—Shortly attenuate.
   Length.—7-10 mm.
   Width.—5-9 mm.
   Margin.—Entire.
   Texture.—Smooth.
   Color.—Upper surface — Young flower: 60A — Old flower: 63A.
   Color.—Lower surface — Young flower: 60B — Old flower: 63B.
   Venation pattern.—Bowed.
   Venation upper surface.—59A.
   Venation lower surface.—199D.
Sepals:
   Quantity.—5 per flower.
   Shape.—Ovate.
   Apex.—Acute.
   Margin.—Entire.
   Length.—4-5 mm.
   Width.—2.5-3.5 mm.
   Color—Upper surface.—146B, with some 63A on apex.
   Color—Lower surface.—146C.
   Texture upper surface.—Smooth; slightly pubescent, hairs have a purple tip (N79B).
   Texture lower surface.—Smooth, glabrous.
Peduncles:
   Length.—1.0-3.5 cm.
   Diameter.—1 mm.
   Color.—144A with much anthocyanin 60A.
   Texture.—Smooth; pubescent, hairs have a purple tip (N79B).
Reproductive organs:
   Androecium.—
   Stamen number.—10, not fused.
   Filament length.—3-5 mm.
   Filament shape.—Threadlike.
   Filament color.—154B.
   Anther shape.—Bi-lobed.
   Anther length.—1.5 mm.
   Anther color.—149A with anhocyanin 63A.
   Color of pollen.—2A.
   Amount of pollen.—Normal.
   Gynoecium.—
   Pistil number.—2.
   Pistil length.—3-4 mm.
   Stigma color.—155A.
   Style color.—1B.
   Ovary color.—150A.
Seed development: Seed development has not been observed to date.
Disease/pest resistance: No disease/pest resistance has been observed to date.

What is claimed is:

1. A new and distinct cultivar of Saxifraga plant named ‘Rockred,’ as substantially illustrated and described herein.
